(19) United States
(12) Patent Application Publication
(10) Pub. No.: US 2018/0014987 A1
(43) Pub. Date: Jan. 18, 2018

(54) SELF-INJURY DETERRENT SLEEVE

(71) Applicant: Cophic Sleeve, LLC, Concord, CA (US)

(72) Inventor: Abraham Karabel, Concord, CA (US)

(21) Appl. No.: 15/210,641

(22) Filed: Jul. 14, 2016

(51) Int. Cl. A61F 15/00; A61F 5/37; A61F 13/06; A61F 13/10

(52) U.S. Cl. CPC A61F 15/008; A61F 13/10; A61F 5/37; A61F 13/06

(57) ABSTRACT

A self-injury sleeve is provided. The presently-disclosed self-injury deterrent sleeves can comprise a sleeve body that is tubular and configured to receive an appendage. The sleeves can further include one or more self-injury deterrent pads provided on an outer side of the sleeve body that are comprised of a material having greater resistance to cutting and abrasion than the sleeve body material, a material that is softer than the sleeve body material, or a combination thereof. The sleeves can be worn by individuals to deter self-cutting and other self-injury.

### TECHNICAL FIELD

The presently-disclosed subject matter generally relates to a self-injury deterrent sleeve. In particular, embodiments of the presently-disclosed subject matter relate to a sleeve for coping with mental health conditions that can deter self-injury.

### INTRODUCTION

Millions of individuals around the world suffer from conditions that affect mental health. In the United States it is estimated that approximately one in five adults suffer from mental health conditions. Depression alone affects approximately 20% to 25% of adults in the United States each year. Other common mental health conditions include, but are not limited to, depression, panic disorders, anxiety disorders, bipolar disorders, substance abuse and addiction, and schizophrenia.

Those suffering from mental health conditions sometimes resort to self-injury or suicide. A portion of self-inflicted injuries are the result of failed suicide attempts. However, individuals may also inflict self-injury to relieve stress, anxiety, or other emotions without the intention or desire to commit suicide. Instead, some individuals inflict self-injury to cope with emotions of feeling numb, pain, distress, depression, negative thoughts, or other unpleasant emotions. Individuals often resort to self-injury because it is private, controllable, and provides short-term relief.

Cutting is a type of self-injury inflicted by those suffering from mental health conditions. Cutting causes physical pain that can distract one's mind from other thoughts or emotions. Cutting also provides a strong physical sensation that can counteract a sensation of feeling mentally numb. For some individuals, cutting also provides a sense of control over one's mind and body.

Psychotherapy is currently used to treat mental health conditions and develop healthy habits for coping with strong emotions. Psychopharmaceuticals are also used to treat certain mental health conditions. High risk subjects, such as those that are likely to engage in self-injury, may further be admitted to psychiatric hospitals or treatment facilities. However, the therapeutic effects of these known treatments are delayed, sometimes by several months or years. Furthermore, these treatments typically require that a subject recognize and consent to treatment, which can be difficult for those who are embarrassed about seeking treatment.

Accordingly, there remains a need for devices and methods for providing short-term emotional relief and deterring self-injury, including cutting. There further remains a need for devices and method that can provide relief from strong emotions quickly and confidentially.

### SUMMARY

Embodiments of the presently-disclosed subject matter meet some or all of the above-identified needs. This Summary describes several embodiments of the presently-disclosed subject matter, and in many cases lists variations and permutations of these embodiments. Such embodiments can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features, which will become evident to those of ordinary skill in the art after a study of the information provided in this document.

The presently-disclosed subject matter includes a self-injury deterrent sleeve that comprises a sleeve body that is tubular and configured to receive an appendage of a user, the sleeve body being comprised of a sleeve body material, and a self-injury deterrent pad (pad) provided on an outer side of the sleeve body, the pad being comprised of a pad material having greater resistance to cutting and abrasion than the sleeve body material.

In some instances the appendage is an arm, and in other instances the appendage is a leg or a finger. In some instances where the appendage is an arm, the sleeve body is configured to extend from a wrist of the arm towards a shoulder of the arm when the arm is received within the sleeve. In other instances, the sleeve body is configured to extend from a wrist to a point along a forearm of an arm. In yet other instances, the sleeve body is configured to extend from a wrist to a point between a shoulder and an elbow of an arm. In instances where the appendage is a finger, the sleeve body is configured to extend along a length of a finger. In some instances where the appendage is a leg, the sleeve body is configured to extend from an ankle of a leg towards a hip of a leg when a leg is received within the sleeve.

In some instances the sleeve body material includes an elastic material. In some instances the sleeve body material includes an elastic writeable material.

In some instances the pad material includes a rubber, a polymer fabric, a plastic, a cotton fabric, or a combination thereof. In some instances the pad protrudes from the outer side of the sleeve body.

In some instances a sleeve body further comprises an opening extending through the sleeve body that is configured to expose a portion of an appendage when the appendage is received within the sleeve body. In some instances the opening is configured to expose an inner wrist of an arm that is received within the sleeve body. The sleeve body can further include an elastic band that extends across the opening.

In some instances the sleeve body further comprises a second self-injury deterrent pad (second pad) provided on the outer side of the sleeve body, the second pad being comprised of a material that is softer than the sleeve body material.

The presently-disclosed subject matter further includes a self-injury deterrent sleeve that comprises a sleeve body that is tubular and configured to receive an appendage of a user, the sleeve body being comprised of a sleeve body material, and a first self-injury deterrent pad provided on an outer side of the sleeve body that is comprised of a first pad material, wherein the first pad material includes a material having greater resistance to cutting and abrasion than the sleeve body material, a material that is softer than the sleeve body material, or a combination thereof.

In some instances the self-injury deterrent sleeve further comprises a second self-injury deterrent pad provided on the outer side of the sleeve body that is comprised of a second pad material, the second pad material including a material having greater resistance to cutting and abrasion than the sleeve body material, a material that is softer than the sleeve body material, or a combination thereof. In some instances the second pad material is different than the first pad material.

In some instances the self-injury deterrent sleeve further comprises a slip-resistant material provided on an inner side of the sleeve body.

The presently-disclosed subject matter further includes a method for deterring self-injury of a subject. In some embodiments the method comprises providing a self-injury deterrent sleeve that includes a sleeve body that is tubular and configured to receive an appendage of a user, the sleeve body being comprised of a sleeve body material, and a self-injury deterrent pad provided on an outer side of the sleeve body, the self-injury deterrent pad being comprised of a pad material that includes a material having greater resistance to cutting and abrasion than the sleeve body material, a material that is softer than the sleeve body material, or a combination thereof. Methods for deterring self-injury can further comprise inserting an appendage of the subject through the sleeve body and permitting the subject to perform one or more actions to deter self-injury.

In some instances, the actions a subject can engage in with the sleeve to deter self-injury include marking the outer side of the sleeve body, replicating an act of self-injury on the self-injury deterrent pad, contacting the self-injury deterrent pad to provide sensory stimulation, or a combination thereof.

### BRIEF DESCRIPTION OF THE DRAWINGS

FIG. 1 shows a side view of a self-injury deterrent sleeve in accordance with an embodiment of the presently-disclosed subject matter.

FIG. 2 shows another side view of the embodied self-injury deterrent sleeve.

FIG. 3 shows a side view of an arm received within the embodied self-injury deterrent sleeve.

FIG. 4 shows a side view of another embodiment of a self-injury deterrent sleeve in accordance with an embodiment of the presently-disclosed subject matter.

FIG. 5 shows a side view of yet another embodiment of a self-injury deterrent sleeve in accordance with an embodiment of the presently-disclosed subject matter.

FIG. 6 shows a side view of an arm received within the embodied self-injury deterrent sleeve depicted in FIG. 5.

### DESCRIPTION OF EXEMPLARY EMBODIMENTS

The details of one or more embodiments of the presently-disclosed subject matter are set forth in this document. Modifications to the exemplary embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes self-injury deterrent sleeves. As discussed herein, embodiments of the sleeves can be used by subjects to cope with mental health conditions. In particular, those with a potential to or that have engaged in self-injury, such as cutting, may utilize embodiments of the sleeves described herein to obtain emotional relief and avoid inflicting self-injury. Furthermore, embodiments of sleeves can provide sensory and other stimulation that aid in deterring self-injury and provide a means for coping with a mental health condition.

As used herein, the term “mental condition” refers to conditions that affect the mind, including psychological and emotional conditions and disorders. Mental health conditions can be associated with desires to inflict self-injury, including intentionally cutting oneself. The term mental health condition thus includes, but is not limited to, any personality disorder, bipolar disorder, depression disorder, obsessive-compulsive disorder, anxiety disorder, psychosis, and the like.

The presently-disclosed sleeve can be worn by a subject on an appendage that may be prone to self-injury. This includes the arms, legs, fingers, and toes of a subject, and the terms “appendage,” “leg,” “arm,” “finger,” and “toe” may be used interchangeably herein. The sleeve itself can provide physical protection against self-injury. Sleeves can also provide emotional relief, distraction, and/or other stimulation that aides in deterring self-injury.

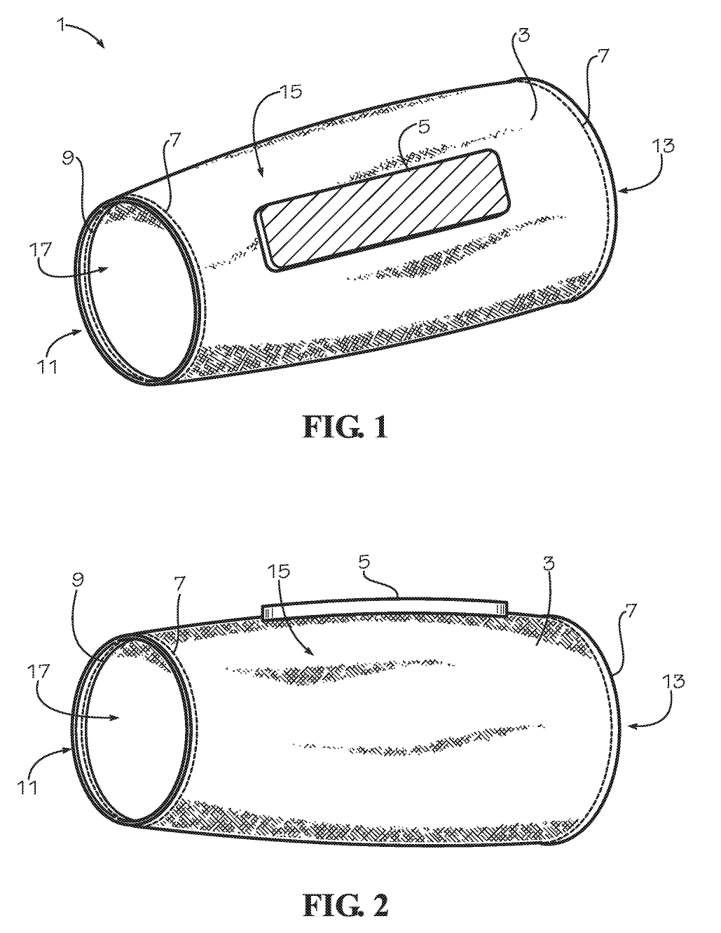

A specific embodiment of a self-injury deterrent sleeve in accordance with an embodiment of the presently-disclosed subject matter will now be described. Unless otherwise stated, any reference to a front, back, left, right, top, bottom, inner, or outer side of the sleeve is made from the perspective shown in FIG. 1. The outer side 15 of the sleeve 1 shown in FIG. 1 remains exposed when the sleeve 1 is worn by a subject.

Looking to FIG. 1, the exemplary self-injury deterrent sleeve 1 includes a sleeve body 3 that is tubular and has two open ends 11, 13. The open ends include a first open end 11 and a second open end 13. The shape and size of the sleeve body 3 can be configured to correspond to the appendage the sleeve 1 is intended to be worn on. The size and shape of the sleeve body 3 are therefore not particularly limited, and can be modified to cover any appendage or other portion of a subject's body that a subject may or has inflicted self-injury to.

The sleeve body 3 is comprised of a sleeve body material. The sleeve body material can include a stretchable and elastic material that permits the sleeve 1 to be worn by different subjects. The stretchable and elastic properties of the sleeve body material can vary depending on the type of material and the intended use of the sleeve 1. In some embodiments a stretchable and elastic material in an extended state can comprise a surface area that is about 10% to about 500% larger than a surface area of the material in a relaxed, upstretched state.

In the embodied sleeve, the sleeve body material is a writeable material. In some embodiments, washable inks can be used on the writeable sleeve material so that the sleeve can be washed, restored, and reused. A subject can mark the sleeve body 3 by writing and illustrating on the sleeve body 3. The term “marking” is used herein to refer to actions such as writing, illustrating, or drawing. Marking can allow a subject to express and release emotions in order to deter the subject from inflicting self-harm.

The elastic properties of the sleeve body material can reduce slippage of the sleeve 1 when it is worn on an appendage. The sleeve body material may also be formed of one or more materials that have non-slip properties in order to further limit slippage of the sleeve 1 when it is worn on an appendage. In some embodiments the sleeve body material includes a plurality of different materials and/or compositions that impart distinct properties to the sleeve body material.

Exemplary materials for the sleeve body can include synthetic materials, non-synthetic materials, or both. Embodiments of materials for sleeve bodies can be comprised of woven fibers and/or non-woven materials. Exemplary sleeve body materials include, but are not limited to, polyurethaneurea, nylon, polyester, cellulose, polyamide, polyolefins, nylon, polyester, cotton, wool, jute, bamboo, polypropylene, polyethylene, polyfluorocarbons, and acrylic materials.

A self-injury deterrent pad 5 (pad) is provided on an outer side 15 of the sleeve body 3. The pad 5 includes a generally rectangular shape and extends across the outer side 15 of the sleeve body 3. As shown in FIG. 2, the longer sides of the rectangular pad 5 extend between the first open end 11 and the second open end 13 of the sleeve 1. Further, because the pad 5 has a thickness that is greater than a thickness of the sleeve body 3, the top side of the pad 5 protrudes from the outer side 15 of the sleeve body 3.

When wearing the sleeve, the size and location of the pad 5 may correspond to an area that a subject may inflict self-injury. However, the size and shape of the pad 5 is not particularly limited, and can vary depending on various factors, including the appendage that the sleeve 1 is to be worn on, the type of self-injury being deterred, the dimensions of a sleeve, and the like.

In some embodiments the pad is rectangular and has a length of about 2 to 8 inches and a width of about 0.5 to about 4 inches. In other embodiments the pad is rectangular and has a length of about 2 to 7 inches and a width of about 1 to about 3 inches. In yet other embodiments the pad is rectangular and has a length of about 3 to 5 inches and a width of about 1 to about 2 inches. In some embodiments the pad is rectangular and has a length of about 4 inches and a width of about 1.5 inches.

In other embodiments the pad does not include a generally rectangular shape. The pad can include a shape of a polygon having any number of sides. In some embodiments the pad includes a shape that is generally circular, elliptical, oval, triangular, or pentagonal. In other embodiments the pad does not include a shape in the form of a polygon, but instead includes an abstract shape that can include two or more sides that are each rounded and/or straight sides.

The pad 5 is comprised of a material that has a greater resistance to cutting and abrasion that the sleeve body 3 material. In this regard, in some instances the material for the pad 5 includes a high denier fabric, including materials having a denier of 200 or more, 400 or more, 600 or more, 800 or more, or 1000 or more. Alternatively or additionally, the material for the pad 5 can include a material selected from KEVLAR®, hardened polymers, plastics, cotton, and combinations thereof. Upon reviewing this document, those of ordinary skill in the art will appreciate other cutting and abrasion resistant materials that can be used for the pad 5.

Still looking to FIGS. 1 and 2, a thread 7 follows the edges of the sleeve body 3 that define the first open end 11 and the second open end 13. The inner side 17 of the sleeve is viewable from the first open end 11 of the sleeve 1, and shows a portion of a hem 9 that is formed by the thread 7. In other embodiments, a hem may or may not be provided depending on, among other things, the material selected for the sleeve body and the method used to manufacture the sleeve body.

Furthermore, in some embodiments a separate slip-resistant material is provided to limit slippage of the sleeve. As shown in FIG. 4, the non-slip material 19 can include a material that forms a circumferential band along the inner side 17 of the sleeve 1. The bands of the non-slip material 19 may be provided adjacent to the first open end 11, the second open end 13, or both. Exemplary non-slip materials include any materials and compositions that resist slippage when worn against the skin, including as silicone, rubber, and other elastic materials.

Non-slip materials can be provided on any area of the inner side 17 of the sleeve 1. For instance, the thread 7 can be formed of a rubber-containing material such that the thread 7 exposed on an inner side 17 of the sleeve 1 limits slippage when the sleeve 1 is worn on an appendage. In other embodiments non-slip materials or substances can be provided on the hem 9 or other portions on the inner side 17 of the sleeve 1. The non-slip materials may or may not be provided circumferentially along the inner side 17 of the sleeve 1. In other embodiments non-slip materials are provided at a plurality of points or regions on the inner side 17 of the sleeve 1.

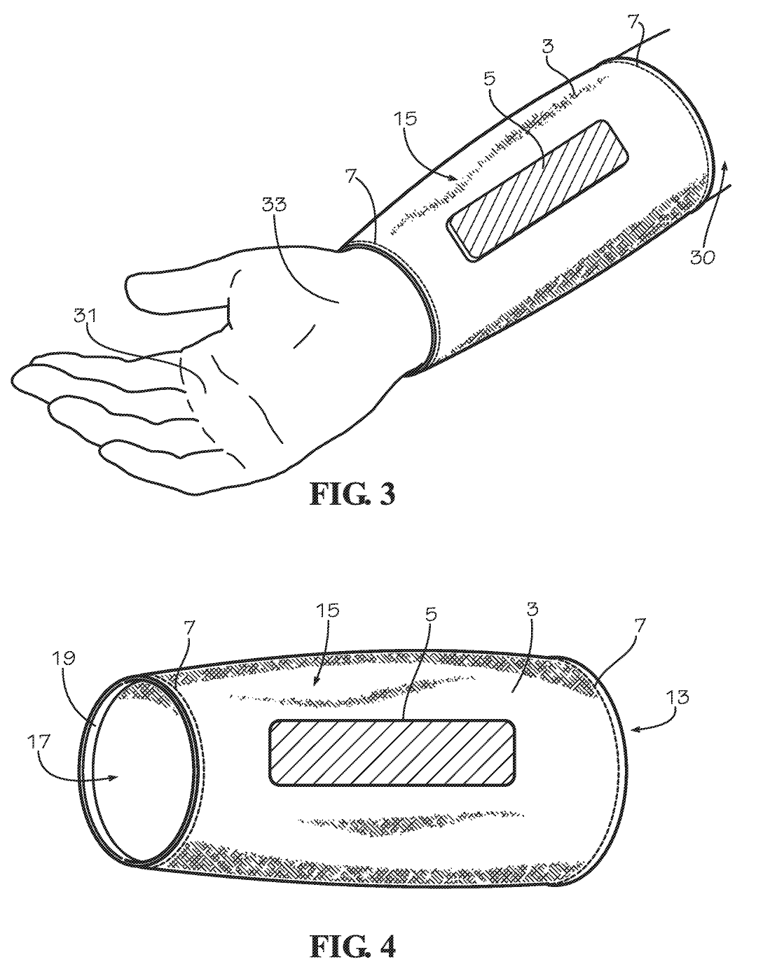

Looking now FIG. 3, a view of the sleeve 1 being worn on an arm (appendage) 30 is shown, wherein the arm 30 is received within the sleeve 1. The sleeve 1 extends from the wrist 33 to a point along the arm 30. The subject's hand 31 remains free and uncovered by the sleeve. A subject can easily utilize the sleeve 1 by inserting their appendage through the first and second open ends 11, 13 of the sleeve body 3. Likewise, a sleeve can be easily removed by a subject by slipping the appendage out of the sleeve.

In other instances, the sleeve preferably extends from the wrist toward the elbow of a subject. In other instances, the sleeve preferably extends from the wrist to a point between the elbow and the shoulder. Similarly, sleeves configured for use on a leg may extend along the shin, the thigh, or any other portion of the leg in order to deter self-injury. In yet other embodiments, a sleeve can extend over one or more fingers or toes of a subject.

The sleeve 1 in FIG. 3 covers a surface of the arm 30 that is typically associated with cutting and self-injury. Likewise, the elongated rectangular pad 5 generally extends along on the outer side 15 of the sleeve body 3 between the elbow and wrist. In particular, the pad 5 covers an area corresponding to the inner forearm on the arm 30 that individuals commonly cut when inflicting self-injury.

The presence of the sleeve 1 and pad 5 alone can deter a subject contemplating cutting or self-injury. For instance, the sleeve 1 can provide a physical layer of protection that causes a subject to think twice or momentarily hesitate before inflicting self-injury. Instead of removing the sleeve 1 to cut oneself, a subject may recognize this impulse and self-correct before inflicting self-injury. Furthermore, a subject may cut, scratch, touch, and/or hit the cutting and abrasion resistant pad 5 to release emotions. These self-injury mimicking actions can provide a subject with an emotional release that deters them from inflicting self-injury.

The length of the sleeve can be altered for particular uses or users, allowing the sleeve to cover any portion of an appendage. Increased coverage may be preferred to increase the extent to which self-injury is deterred. Contrarily, it may be preferable to minimize the length of the sleeve so that the sleeve can be concealed more easily when worn. Similarly, the size and shape of the pad can be modified depending on the appendage the sleeve is worn on, the size of the sleeve, and the type of self-injury being deterred.

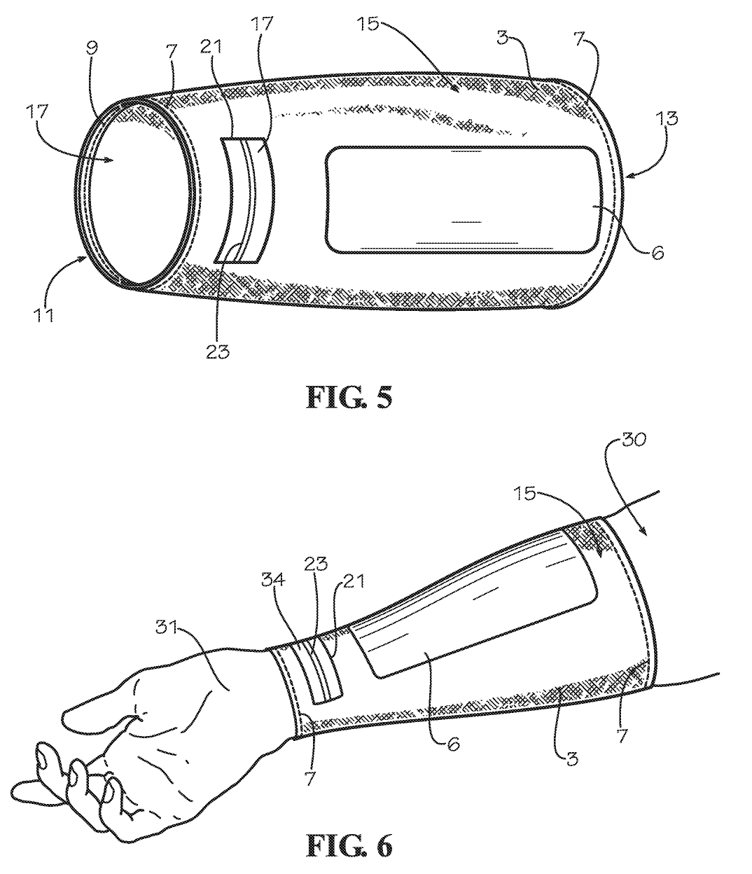

Looking now to FIG. 5, another embodiment of the presently-disclosed sleeves is shown. The sleeve includes a tubular sleeve body 3 having a first open end 11 and a second open end 13. A self-injury deterrent pad 6 (second pad) is provided on an outer side 15 of the sleeve body 3. The pad 6 is comprised a material that is softer than the sleeve body material. In some embodiments the material for the pad 6 can include a lower denier than a sleeve body material. In some embodiments the material for the pad 6 includes asoft material such as a fleece material, silk, or the like.

The size, shape, and location of the pad 6 are not particularly limited. In some instances the pad 6 corresponds to an area prone to self-injury when the sleeve is worn. In this regard, the statements made herein regarding the size, shape, and location of a pad 5 (first pad) are also applicable to certain embodiments of a pad 6 (second pad).

A subject may touch or rub the soft material of the pad 6 to promote and stimulate sensation and other emotions. Tactile sensory information from contacting or touching the pad 6 can aid in coping with certain emotions so as to deter potential acts of cutting and self-injury. As with the first pad 5, in some embodiments the second pad 6 is positioned so that it corresponds to an area typically associated with cutting and self-injury when the sleeve 1 is worn.

The sleeve 1 shown in FIG. 5 further includes an opening 21 that extends through the sleeve body 3. When the sleeve with an opening 21 is worn, a portion of the appendage is exposed from the opening 21. The opening 21 can be sized so that tactile senses can be stimulated through the opening 21 while still providing sufficient coverage to deter self-injury. For instance, a subject can touch, heat, or cool (e.g., apply ice) the appendage through the opening 21 to promote senses and deter self-injury.

In some embodiments the opening is generally rectangular and has a length of about 0.5 to 4 inches and a width of about 0.5 to about 4 inches. In other embodiments the opening is rectangular and has a length of about 1 to 3 inches and a width of about 1 to about 3 inches. In yet other embodiments the opening is rectangular and has a length of about 1 to 2 inches and a width of about 1 to about 2 inches. In some embodiments the opening is rectangular and has a length of about 1.5 inches and a width of about 1.25 inches.

In other embodiments the opening does not include a generally rectangular shape. In some embodiments the opening includes a shape that is generally circular, elliptical, oval, triangular, or pentagonal. In other embodiments the opening does not include a shape in the form of a polygon, but instead includes an abstract shape that can include two or more sides that are each rounded and/or straight sides.

An elastic band 23 extends across the center of the opening 21 in a direct that corresponds to the longer side of the opening 21. The elastic band 23 can be pulled and snapped on the skin to safely release emotional tension. As shown in FIG. 6, the sleeve is configured so that the opening 21 is positioned over an inner wrist 34 when the sleeve 1 is worn. Likewise, the elastic band 25 crosses over the inner wrist 34 when the sleeve 1 is worn. A subject can use the opening 21, elastic band 23, and/or pad 6 to provide sensory stimulation and release strong emotions. In other embodiments any number of elastic bands can extend in any direction across an opening provided in a sleeve.

A subject can wear a self-injury deterrent sleeve in times of need, and can remove the sleeve once the need subsides. The sleeve thereby permits subjects to obtain short-term emotional relief to deter them from inflicting self-harm. Users will appreciate that the sleeves provide a non-permanent solution for coping with mental health conditions and strong emotions.

The sleeves are also beneficial because they can be used by those without any medical training, permitting their use even when a nurse, doctor, or other mental health professional is not available to help a subject cope with their desire to inflict self-injury.

Embodiments of the sleeves described herein can also be used at times where traditional treatments are ineffective or unavailable. For instance, a subject can use a sleeve to deter self-injury when a strong impulse to inflict self-injury arises between psychotherapy sessions or when psychotherapy has not yet completely abated desires to inflict self-injury in the subject. Similarly, subjects that intentionally or unintentionally do not take medications for coping with desires to inflict self-injury can use a sleeve to immediately deter acts of self-injury. Accordingly, in some instances a sleeve can be used without additional treatment to deter acts of self-injury, and in other instances a sleeve can be used in conjunction with psychopharmaceuticals and/or psychotherapy to deter acts of self-injury.

The principal features of the presently-disclosed subject matter that are described in the present disclosure may be employed in various embodiments without departing from the scope of the present invention. Those of ordinary skill in the art will recognize numerous equivalents to the specific apparatuses and processes described herein. Such equivalents are considered to be within the scope of the presently-disclosed subject matter and are covered by the claims.

For instance, embodiments of sleeves can include any number and combination of the self-injury deterring elements, including pads, openings, and elastic bands, described herein. In a specific embodiment a sleeve includes a first self-injury deterrent pad and a second self-injury deterrent pad, the pads being made of any of the materials disclosed herein. In some embodiments a first pad and a second pad are formed of different materials. Other specific embodiments of sleeves can include any number and size of openings on a sleeve body, and one or more elastic bands can extend across any of the openings.

The presently-disclosed subject matter can comprise, consist of, or consist essentially of the elements and features of the embodiments described herein, as well as any additional or optional components or limitations described herein or otherwise useful.

As used herein, the term “about,” when referring to a value or measurement is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate.

All references to singular characteristics or features of the present disclosure shall include the corresponding plural characteristic(s) or feature(s) and vice versa, unless otherwise specified or clearly implied to the contrary by the context in which the reference is made.

### NUMBERED LIST OF ELEMENTS

1—self-injury deterrent sleeve (sleeve)

3—sleeve body

5—self-injury deterrent pad (first pad)

6—self-injury deterrent pad (second pad)

7—thread

9—hem

11—first open end

13—second open end

15—outer side

17—inner side

19—non-slip material

21—opening

23—elastic band

30—appendage

31—hand

33—wrist

34—inner wrist

What is claimed is:

1. A self-injury deterrent sleeve, comprising:
a sleeve body that is tubular and configured to receive an appendage, the sleeve body being comprised of a sleeve body material; and
a pad provided on an outer side of the sleeve body, the pad being comprised of a pad material having greater resistance to cutting and abrasion than the sleeve body material.

2. The sleeve of claim 1, wherein the appendage includes an arm or a leg.

3. The sleeve of claim 2, wherein the sleeve body is configured to extend from a wrist to a point along a forearm of the arm when the arm is received within the sleeve body.

4. The sleeve of claim 2, wherein the sleeve body is configured to extend from a wrist to a point between a shoulder and an elbow of the arm when the arm is received within the sleeve body.

5. The sleeve of claim 1, wherein the sleeve body material includes an elastic material.

6. The sleeve of claim 1, wherein the pad protrudes from the outer side of the sleeve body.

7. The sleeve of claim 1, wherein the pad material includes a rubber, a polymer fabric, a plastic, a cotton fabric, or a combination thereof.

8. The sleeve of claim 1, further comprising an opening that extends through the sleeve body, the opening being configured to expose a portion of the appendage when the appendage is received within the sleeve body.

9. The sleeve of claim 8, further comprising an elastic band that extends across the opening.

10. The sleeve of claim 1, further comprising a second pad provided on the outer side of the sleeve body, the second pad being comprised of a material that is softer than the sleeve body material.

11. A self-injury deterrent sleeve, comprising:
a sleeve body that is tubular and configured to receive an appendage, the sleeve body being comprised of a sleeve body material; and
a first self-injury deterrent pad provided on an outer side of the sleeve body that is comprised of a first pad material;
wherein the first pad material includes a material having greater resistance to cutting and abrasion than the sleeve body material, a material that is softer than the sleeve body material, or a combination thereof.

12. The sleeve of claim 11, further comprising a second self-injury deterrent pad provided on the outer side of the sleeve body that is comprised of a second pad material;
wherein the second pad material includes a material having greater resistance to cutting and abrasion than the sleeve body material, a material that is softer than the sleeve body material, or a combination thereof; and
wherein the second pad material is different than the first pad material.

13. The sleeve of claim 11, further comprising a slip-resistant material provided on an inner side of the sleeve body.

14. The sleeve of claim 11, wherein the appendage is an arm, and wherein the sleeve body is configured to extend from a wrist of the arm towards a shoulder of the arm when the arm is received within the sleeve body.

15. The sleeve of claim 11, wherein the appendage is a leg, and wherein the sleeve body is configured to extend from an ankle of the leg towards a hip of the leg when the leg is received within the sleeve body.

16. The sleeve of claim 11, wherein the sleeve body material includes an elastic writeable material.

17. The sleeve of claim 11, further comprising an opening that extends through the sleeve body, the opening being configured to expose a portion of the appendage when the appendage is received within the sleeve body.

18. The sleeve of claim 17, wherein the appendage is an arm, and wherein the opening is configured to expose an inner wrist of the arm when the arm is received within the sleeve body.

19. The sleeve of claim 17, further comprising an elastic band that extends across the opening.

20. A method for deterring self-injury of a subject, comprising:
providing a self-injury deterrent sleeve that includes
a sleeve body that is tubular and configured to receive an appendage of the subject, the sleeve body being comprised of a sleeve body material; and
a self-injury deterrent pad provided on an outer side of the sleeve body, the self-injury deterrent pad being comprised of a pad material that includes a material having greater resistance to cutting and abrasion than the sleeve body material, a material that is softer than the sleeve body material, or a combination thereof;
inserting the appendage of the subject through the sleeve body;
permitting the subject to perform one or more actions to deter self-injury selected from:
marking the outer side of the sleeve body;
replicating an act of self-injury on the self-injury deterrent pad; and
contacting the self-injury deterrent pad to provide sensory stimulation in the subject.
